(19) United States
(12) Patent Application Publication
(10) Pub. No.: US 2001/0008887 A1
(43) Pub. Date: Jul. 19, 2001

(54) METHOD FOR DETECTION OF PATHOGENS IN FOOD

(76) Inventors: PRABHAKARA V. CHOUDARY, DAVIS, CA (US); CHRISTOPHER M. GOODING, NORWICH (GB)

Correspondence Address: HANA VERNY, PETERS VERNY JONES & BIKSA, 385 SHERMAN AVENUE, SUITE 6, PALO ALTO, CA, 94306

(21) Appl. No.: 09/308,106

(22) PCT Filed: Nov. 4, 1996
(86) PCT No.: PCT/US96/17535

§ 371 Date: None

(51) Int. Cl. A61K 31/665; A01N 57/00; G01N 33/02

(52) U.S. Cl. 514/100; 436/20

(57) ABSTRACT

The invention provides a method for the specific detection of a target pathogen in a sample. The method of the invention comprises, as a first step, incubating the sample under sufficient conditions to enrich the target pathogen population in the sample. As a second step, the target pathogen is selected by immunoselection from the sample enriched in the first step. The selection of the target pathogen is followed by amplification of a nucleic acid sequence specific to the target pathogen from the target pathogen so selected. The amplified nucleic acid sequence is then detected, the presence of the amplified nucleic acid sequence indicating the presence of the target pathogen. By following the method of the invention, a single colony-forming unit (CFU) of bacterial pathogen can be detected in a contaminated food product in 8 hours or less. The invention provides improved tools for rapid and early detection of pathogens, including E. coli O157:H7, and Salmonella strains: S. agona; S. anatum; S. enteritidis; S. havana; S. krefeld; S. lilee; S. melegredis; S. montevideo; S. munster; S. newport, S. saintpaul; S. schwarzengrund; S. tennessee; S. typhimurium and S. worthington, among others. In one embodiment, the invention provides a method for the detection of a target pathogen in a food sample. This method can be useful for screening food products for the presence of contaminating organisms to minimize the risk of food poisoning. In another embodiment, the invention provides a method for the detection of a target pathogen in a patient sample. This method can be useful for diagnosing infection by a microbial pathogen.

### BACKGROUND

Salmonellosis is one of the oldest and most common food poisoning syndromes, with an overall mortality rate of up to 0.2%. An estimated 4 million cases of Salmonella infection are reported each year in the US alone (Doyle, M. P. & Cliver, D. O. (1990) in Foodborne Diseases, Cliver, D. O., ed., 185-205, Academic Press, New York/ London). Unlike in the past, when S. typhimurium was the principal causative agent of Salmonellosis, the past decade has witnessed S. enteritidis claim the top spot. With animal intestinal tract as its natural habitat, Salmonella enters the human food chain mainly through foods of animal origin, including poultry, beef, pork, cheese, mayonnaise, baby formulas and canned sea food.

In response to the mounting public concern the US administration has recently announced plans for a drastic reform in the antiquated methods used by the USDA-FSIS based on “see-smell-and-feel” approach for certification of microbiological safety of fresh meat for human consumption. The soaring numbers of fatalities being reported currently on a daily basis (especially from Japan), however, are caused by a newly emerging foodborne pathogen, Escherichia coli O157:H7. Though recent in emergence, because of its portending ability to become a “serial killer” in a potentially epidemic scenario, E. coli O157:H7 has come to occupy the center stage, leading to the development of a spate of techniques each with specific advantages over the other in terms of rapidity, sensitivity, specificity, cost economy or ease of use.

Rapid and reliable techniques that facilitate detection of foodborne microbial pathogens at early stages of contamination are key to the development of effective prevention and control strategies. The recent dramatic increase in Escherichia coli O157:H7 outbreaks has served to highlight the paramount importance of developing new methods that allow faithful implementation of hazard analysis and critical control points (HACCP) principles all along the food chain.

The enterohemorrhagic Escherichia coli (EHEC) O157:H7 strain, a prominent member of the verotoxin-producing E. coli (VTEC), is increasingly associated with major outbreaks of food- and water-borne infectious disease in humans. The virulence of EHEC strains is in part due to the production of the verotoxins, shiga-like-toxin 1 (SLT-I) and shiga-like-toxin 2 (SLT-II) made up of an A (active) subunit and a B (binding) subunit (Karmali, M. A., 1989, Clinical Microbiology Reviews 2:15-38). Syndromes arising from infection with EHEC include bloody diarrhea, haemolytic uraemia syndrome (HUS) and haemorrhagic colitis. The importance of this infection is emphasised by evidence that E. coli O157:H7-associated HUS can lead to the death of up to 8% of infected children (Brandt, J. R., et al., 1994, Journal of Pediatrics 125:519-26).

Most human infections caused by E. coli O157:H7 are foodbome, and foods of bovine origin have been identified as the principal carriers of the pathogen in a majority of the outbreaks. Undercooked ground beef has been the primary vehicle for pathogen transmission in most major outbreaks, but other foods including raw (unpasteurized) milk, potatoes and yoghurt have also been epidemiologically implicated (Doyle, M. P., 1991, International Journal of Food Microbiology 14:289-301). The largest recorded outbreak due to contaminated milk occurred recently in Scotland, infecting 100 people and hospitalising 33 (Upton, P. & Coia, J. E., 1994, Lancet 344:1015). While ice cream has not yet been directly implicated in outbreaks of E. coli O157:H7, it is recognised as a vehicle for infection by other E. coli strains (Rothwell, J., 1990, Dairy Microbiology Volume 2, Ed Robinson, R. K., Elsevier Applied Science. London. 1-40.), and thus has a strong potential to transmit EHEC. Ice cream can be contaminated by faulty manufacture or bad post-process handling and given its cryo-preserving properties EHEC may survive subsequent freeze-thaw cycles very well.

Escherichia coli O157:H7 was identified conclusively as a food pathogen in 1982, with the demonstration of its causal association with 2 outbreaks of hemorrhagic colitis, following the consumption of hamburgers (Riley, L. W., et al., 1983, New England Journal of Medicine 208:681-85; Wells, J. G., et al., 1983, Journal of Clinical Microbiology 18:512-520). Since then, it has been linked with many other outbreaks in the USA, Canada and the UK and is now the most frequently isolated diarrheagenic type of E. coli in North America (Tarr, P. I., 1995, Clinical Infectious Diseases 20:1-8). A major epidemic of E. coli O157:H7, with over 9,000 cases and 11 deaths, has also occurred recently in Japan, although a conclusive determination of the precise source of infection has yet to be made (Swinbanks, D., 1996, Nature 382:290).

Most outbreaks have been associated with the consumption of undercooked beef or raw milk. A survey of retail uncooked meats has revealed the presence of E. coli O157:H7 in 3.7% of fresh beef, 1.5% of pork and poultry and 2% of lamb (Doyle, M. P. and Schoeni, J. L., 1987, Applied Environmental Microbiology 53:2394-2396). To address epidemiological issues and for developing effective management strategies, such as HACCP, for the prevention and control of this deadly pathogen, a rapid and sensitive method is required.

Several methods have been developed for the detection and isolation of foodborne pathogens, some of which can be used in the detection of E. coli O157:H7 in dairy foods. The conventional methods involve microbiological culturing and isolation of the pathogen, followed by isolate-confirmation by biochemical and/or serological tests. These are time-consuming and labour-intensive, and are thus costly (Meng, J., et al., 1994, Trends in Food Science and Technology 5:179-185). For example, testing for inability to ferment sorbitol, a characteristic feature of EHEC, does not always provide an unambiguous result in tests to distinguish EHEC strains from non-EHEC strains. Immunoassays are more rapid and less labour-intensive than culture methods, but they are not robust enough to serve as independent procedures. Further, the interpretation of immunoassay test results is often marred by false positives, requiring further confirmation (Meng, J., et al., 1994, Trends in Food Science and Technology 5:179-185).

Several new commercial procedures, based on pathogen-specific gene probes and nucleic acid-amplification technologies, have been introduced into the market. However, even these advanced techniques take days (at least two) rather than hours, and lack the required specificity to identify the pathogen at a sub-genus level. Recently, there has been increased interest in more rapid and sensitive techniques such as polymerase chain reaction (PCR), which, by an iterative cycle of DNA synthesis, can generate up to 10⁷ copies of a target stretch of DNA sequence in less than 3 h, using a thermostable DNA polymerase and specific oligonucleotide primers. PCR procedures of various formats and combinations have been developed based on VT1 and VT2 genes as templates, and are used by several investigators for detection of verotoxigenic E. coli (VTEC).

Gannon et al. (Applied and Environmental Microbiology 58:3809-3815) have used PCR to detect Shiga-like toxin-producing E. coli, SLTEC (an alternative nomenclature for verocytotoxic E. coli, VTEC) in ground beef. In a total of 15 h (a 6-h culture enrichment step, followed by a 9-h isolation of DNA, PCR and agarose gel electrophoresis) they achieved the sensitivity of detection of 1 cfu per gram of meat. Begum and Jackson (Begum, D. and Jackson, M. P., 1995, Molecular and Cellular Probes 9:256-264) have used PCR to detect Shiga-like toxin-producing E. coli directly in beef homogenates, with the detection limit of 30 SLTEC/ml, and have done away with pre-enrichment and DNA isolation. Neither the method of Gannon et al. nor that of Begum and Jackson, however, is specific for the O157:H7 strain of E. coli. Others have used immunomagnetic separation (IMS) to selectively capture E. coli O157:H7 from enrichment broths. In spite of the individual merits of each of these methods over the earlier methods, a wide gap remains in the overall efficiency of the methods available and those required for effective prevention and control strategies.

We have devised a novel strategy to solve this problem of inefficiency by assembling a synergistic combination of microbiological and immunological approaches with nucleic acid amplification-based procedures. The modules of this combination can be readily assembled in a desired format, combining only the required steps, to fit a specific contamination situation. Using the new method, we have been able to reduce the detection time considerably, to 8 h to detect a single colony forming unit (cfu) of E. coli O157:H7 in 1 g of raw milk, ice cream or ground meat. We can reduce the detection time further using primer- or probe reporters with fluorescent or chemiluminiscent labels.

### SUMMARY OF THE INVENTION

The invention provides a method for the specific detection of a target pathogen in a sample. The method of the invention comprises, as a first step, incubating the sample under sufficient conditions to enrich the target pathogen population in the sample. As a second step, the target pathogen is selected by immunoselection from the sample enriched in the first step. The selection of the target pathogen is followed by amplification of a nucleic acid sequence specific to the target pathogen from the target pathogen so selected. The amplified nucleic acid sequence is then detected, the presence of the amplified nucleic acid sequence indicating the presence of the target pathogen. By following the method of the invention, a single colony-forming unit (cfu) of bacterial pathogen can be detected in a contaminated food product in 8 hours or less. The invention provides improved tools for rapid and early detection of pathogens, including E. coli O157:H7, and Salmonella strains: S. agona; S. anatum; S. enteritidis; S. havana; S. krefeld; S. lilee; S. melegredis; S. montevideo; S. munster; S. newport; S. saintpaul, S. schwarzengrund; S. tennessee; S. typhimurium and S. worthington, among others.

### BRIEF DESCRIPTION OF THE DRAWINGS

FIG. 1 shows a primer set that amplifies a 215 base pair (bp) coding sequence of the A subunit of SLT-I and a 212 bp coding sequence of the A subunit of SLT-II.

FIG. 2 shows primer sets used for PCR-detection of Salmonella.

FIG. 3 shows the results of gel electrophoresis on samples of raw milk following a 4 hour enrichment, immunomagnetic separation and PCR amplification of fragments of the shiga-like toxin genes.

FIG. 4 shows the results of electrophoretic detection of the SLT-gene amplification products from the immuno-PCR of ground beef samples, spiked with Escherichia coli O157:H7.

### DETAILED DESCRIPTION OF THE INVENTION

### Definitions

As used herein, “target pathogen” means a pathogenic microorganism. In one embodiment, the target pathogen is a microbial pathogen. In another embodiment, the target pathogen is a fungus, such as yeast. In another embodiment, the target pathogen is a gram-positive bacterium. Examples of gram-positive bacteria as target pathogens include, but are not limited to, Listeria and Clostridium tyrobutyricum, the latter of which can contaminate semi-soft and hard cheeses. In yet another embodiment, the target pathogen is a gram-negative bacterium, such as a gram-negative enterobacterium. Examples of gram-negative bacteria as target pathogens include, but are not limited to: lactic acid bacteria; Shigella; Vibrio, such as V. cholerae and V. haemolytica; Escherichia coli, such as Escherichia coli O157:H7; and Salmonella such as S. agona; S. anatum; S. enteritidis; S. havana; S. krefeld; S. lilee; S. melegredis; S. montevideo; S. munster; S. newport; S. saintpaul; S. schwarzengrund; S. tennessee; S. typhimurium and S. worthington. In another embodiment, the target pathogen is Cryptosporidium, which can contaminate drinking water.

As used herein, a “sample” means any material that contains, or potentially contains, biological material which could be contaminated by the presence of a pathogenic microorganism. Examples of samples for use in accordance with the invention include, but are not limited to, food samples, patient samples (e.g., feces or body fluids, such as urine, blood or cerebrospinal fluid) water, such as drinking water or other fluids. Examples of a food sample include, but are not limited to: dairy products such as cheese, yogurt, ice cream or milk, including raw milk; meat such as beef, pork, minced meat, turkey, chicken or other poultry products; ground meat such as ground beef, ground turkey, ground chicken, ground pork; eggs; produce, including fruits and vegetables; peanut butter; seafood products including oysters, pickled salmon or shellfish; or juice, such as fruit or vegetable juice.

As used herein, “incubating” means maintaining the sample in conditions that enhance the proliferation of the target pathogen, thereby enriching the target pathogen population in the sample. The conditions that enhance the proliferation of the target pathogen will vary with the target pathogen and are known to those skilled in the art. In one embodiment, incubating is by culturing, such as culturing under standard conditions. The culture media may be selective or non-selective. Examples of culture media for use in the method of the invention include, but are not limited to, pre-enrichment broth, selective enrichment broth, chloryl broth, selective agromedia or tryptic soy broth. Guidance on the selection of suitable media for different organisms may be found in AOAC International, “Food and Drug Administration Bacteriological Analytical Manual,” 7th Edition, pp. 1-529, 1992.

As used herein, “selecting the target pathogen” means isolating target pathogen-rich material from target pathogen-poor material, thereby obtaining a sufficiently enhanced concentration of the target pathogen so that reliable, reproducible detection of the target pathogen can be achieved. In one embodiment, selection is effected by fluorescence-activated cell sorting (facs). In another embodiment, the cells can be sorted on the basis of bioluminescence (Kricka, L. J., 1995, Analytical Chemistry 67(12):499R-502R; Tu, S. C. and Mager, H. I., 1995, Photochemistry and Photobiology 62(4):615-24; Duffy, G. et al., 1995, Applied & Environmental Microbiology 61(9):3463-5). In another embodiment, selection is effected by immunoselection using an antibody or fragment thereof that specifically recognizes, binds and captures the target pathogen. Examples of immunoselection include, but are not limited to, selection using paramagnetic beads coated with the antibody or fragment thereof that specifically recognizes, binds and captures the target pathogen.

As used herein, “amplification of a nucleic acid sequence” means any method whereby a specific nucleic acid sequence may be amplified. Examples of amplification methods include, but are not limited to: polymerase chain reaction (PCR), including DNA- or RNA-based PCR, RT-PCR, immunoPCR; ligase chain reaction (LCR), self-sustained sequence replication or Q beta replicase assay.

### Methods of the Invention

The invention provides a method for the specific detection of a target pathogen in a sample. The method of the invention comprises, as a first step, incubating the sample under sufficient conditions to enrich the target pathogen population in the sample. As a second step, the target pathogen is selected from the sample enriched in the first step. The selection of the target pathogen is followed by amplification of a nucleic acid sequence specific to the target pathogen from the target pathogen so selected. The amplified nucleic acid sequence is then detected, the presence of the amplified nucleic acid sequence indicating the presence of the target pathogen. By following the method of the invention, a single colony-forming unit (cfu) of bacterial pathogen can be detected in a contaminated food product in 8 hours or less. The invention provides improved tools for rapid and early detection of pathogens, including E. coli O157:H7, and Salmonella strains: S. agona; S. anatum; S. enteritidis; S. havana; S. krefeld; S. lilee; S. melegredis; S. montevideo; S. munster; S. newport; S. saintpaul; S. schwarzengrund; S. tennessee; S. typhimurium and S. worthington, among others.

In one embodiment, the invention provides a method for the detection of a target pathogen in a food sample. This method can be useful for screening food products for the presence of contaminating organisms to minimize the risk of food poisoning. In another embodiment, the invention provides a method for the detection of a target pathogen in a patient sample. This method can be useful for diagnosing infection by a microbial pathogen.

### A. Incubation Step

In one embodiment, the incubation step of the method of the invention comprises culturing the food sample in tryptic soy broth at 37° C. The conditions for optimizing the development and/or proliferation of the target pathogen will vary with the target pathogen and are known to those skilled in the art (AOAC International, Food and Drug Administration Bacteriological Analytical Manual, 7th Edition, pp. 1 -529, 1992). In one embodiment, incubating is by culturing under standard conditions. The culture media may be selective or non-selective. The incubation time, temperature, media and other conditions will vary with the nature of the sample and the target pathogen, and are selected to culminate in a final titer of the target pathogen that is sufficient for subsequent detection of the amplified nucleic acid sequence.

One factor in determining incubation conditions, such as time, is the doubling time of the target pathogen. For E. coli the doubling time is approximately 20 minutes. To detect 1 cfu of E. coli O157:H7 by agarose gel electrophoresis, for example, an incubation of about 4 hours in tryptic soy broth at 37° C. is sufficient. For a higher detection criterion, such as 100 cfu, the incubation time can be reduced to about 2 hours. The incubation time may be further reduced if a more sensitive method of detection is used. Examples of more sensitive methods of detection include, but are not limited to, nucleic acid assays wherein the nucleic acid is labeled with a fluorescent intercalating dye, which allows for measurement of the amplification of nucleic acid sequences in real time (Burg, J. L. et al., 1995, Analytical Biochemistry 230(2):253-72). One example of a fluorescent intercalating reporters dye is propidium iodide. Examples of other nucleic acid markers that can be used to accelerate detection of amplified nucleic acid sequences include, but are not limited to, a chemiluminescent marker, immuno-affinity tags such as c-myc, affinity tags such as cellulose-binding domain, streptag, biotin/streptavidin or any whole or part macromolecule with a matching fit, reporter enzymes with chromogenic, luminescent, fluorescent or other tracer capabilities, or adducts that confer special and distinct properties on the target (G. H. Keller, M. M. Manak, DNA Probes, 2nd Edition, Stockton Press, New York, N.Y., pp. 1-659, 1993).

### B. Pathogen Selection

In one embodiment, the pathogen selection step of the method of the invention comprises immunoselection using an antibody or fragment thereof that specifically recognizes, binds and captures the target pathogen. Examples of immunoselection include, but are not limited to, immunomagnetic separation using paramagnetic beads coated with the antibody or fragment thereof that specifically recognizes, binds and captures the target pathogen. Examples of antibodies that specifically recognize, bind and capture E. coli O157:H7 include, but are not limited to, Dynabeads anti-E. coli O157:H7 (Dynal As, Oslo, Norway). A variety of antibodies for use in the method of the invention are commercially available (Accurate Chemical and Scientific Corporation, Westbury, N.Y.; Affinity Bioreagents Inc., Golden, Colo.; Pierce, Rockford, Ill.).

Where antibodies are not yet commercially available, techniques for producing them are known in the art (Choudary P V, H A lee, B D Hammock, M R A Morgan, Recombinant antibodies: new tools for immunoassays, in New Frontiers in Agrochemical Immunoanalysis, D A Kurtz, J H Skerrit, L Stanker (Eds), AOAC International, Washington, D.C., pp. 171-185; Hermanson, G. T., Bioconjugate Techniques, Academic Press, New York, N.Y., pp. 1-659, 1996). For example, one can raise antibodies in rabbits by innoculating them with formalin-treated bacteria, which is currently being done with Vibrio haemolytica. The antibody or fragment thereof for use in the immunomagnetic separation can be attached to the paramagnetic beads according to methods known in the art (Hermanson, G. T., Bioconjugate Techniques, Academic Press, New York, N.Y., pp. 1-659, 1996).

The paramagnetic beads for use in immunoselection can comprise, for example, metal, glass, sugar, cellulose or polymer. The binding affinities will vary with the material used for the beads.

### C. Amplification of Nucleic Acid Sequences

In one embodiment, the amplification of a nucleic acid sequence specific to the target pathogen is by polymerase chain reaction (PCR). Examples of primers that can be used for amplification of nucleic acid sequences specific to E. coli O157:H7 include, but are not limited to, a primer set that amplifies a 215 base pair (bp) coding sequence of the A subunit of SLT-I and a 212 bp coding sequence of the A subunit of SLT-II, such as:

A: ^{5′}-ATACAGAG(GA)G(GA)ATTTCGT-^{3′}

B: ^{5′}-TGATGATG(AG)CAATTCAGTTAT-^{3′}.

Examples of primers that can be used for amplification of nucleic acid sequences specific to Salmonella include, but are not limited to, any of the following primer sets:

#1 ST 11: ^{5′}AGCCAACCATTGCTAAATTGGCGCA^{3′}

ST 15: ^{5′}GGTAGAAATTCCCAGCGGGTACTG^{3′};

#2 1: ^{5′}TTATTAGGATCGCGCCAGGC^{3′}

2: ^{5′}AAAGAATAACCGTTGTTCAC^{3′};

#3 a: ^{5′}TGCCTACAAGCATGAAATGG^{3′}

b: ^{5′}AAACTGGACCACGGTGACAA^{3′};

#4 a: ^{5′}GATCATCCATTCGGCATTAAACA^{3′}

b: ^{5′}TTCTCAGCGACGGAAGGGTAAATC^{3′}; or

#5 S 18: ^{5′}ACCGCTAACGCTCGCCTGTAT^{3′}

S19: ^{5′}AGAGGTGGACGGGTTGCTGCCGTT^{3′}.

The invention includes methods in which other primers that are at least 80% homologous to the target nucleic acid sequence of the pathogen are used for the amplification of nucleic acid sequences specific to the target pathogen. The target nucleic acid sequence of the target pathogen can be any nucleic acid sequence specific to the target pathogen, including sequences from the chromosomal/organellar genome(s), plasmid regions, lysogenic phages, genomic sequences, ribosomal genes, among others. The invention also includes methods in which other primers derived from the sequences of SLT-I and SLT-II that encompass at least about a 100 bp sequence for the amplified product are used for the amplification of nucleic acid sequences specific to the target pathogen. The invention also includes methods in which other primers that are derived from the sequences of other toxins produced by microbial pathogens are used for the amplification of nucleic acid sequences specific to the target pathogen.

The person skilled in the art can select primers for amplification of nucleic acid sequences specific to the target pathogen from the published literature or from public databases. The factors affecting the identification of suitable primers for this purpose will vary with the nature of the target pathogen. For example, if specific detection of a particular strain of pathogen is desired, such as E. coli O157:H7, primers will be selected for their specificity for the particular strain. If detection of a group of pathogens is desired, such as a variety of Salmonella strains, then primers will be selected for their ability to amplify nucleic acid sequences of any member of the group of pathogens. Thus, once the target pathogen has been identified, the skilled person can select the appropriate primers.

In one embodiment, the amplification of nucleic acid sequences specific to the target pathogen is by PCR. In another embodiment, the amplification of nucleic acid sequences specific to the target pathogen is by RNA PCR. In another embodiment, the amplification is by immunoPCR. In another embodiment, the amplification is by cascade amplification. In another embodiment, the amplification is by ligase chain reaction (LCR; Wiedmann, M. et al., 1994, PCR Methods & Application 3(4):S51-64; Zebala, J. A. and Barany, F., 1993, J. Clin. Gastroenterol. 17(2):171-5; Laffler, T. G. et al., 1993, Annales de Biologie Clinique 51(9):821-6). In another embodiment, the amplification is by new strand displacement assay. In another embodiment, the amplification is by self-sustained sequence replication. In another embodiment, the amplification is by Q beta replicase assay (Tyagi, S. et al., 1996, PNAS 93(11):5395-400; Burg, J. L., 1995, Analytical Biochemistry 230(2):263-72). Different methods of nucleic acid amplification available have been reviewed by: Abramson, R. D. and Myers, T. W., 1993, Current Opinion in Biotechnology 4(1):41-7; Hagen-Mann, K. and Mann W., 1995, Exper. and Clin. Endocrinol. and Diabetes 103(3):150-5; and Pfeffer, M. et al., 1995, Veterinary Research Communications 19(5):375-407.

To access DNA of some pathogens for the amplification step, it may be necessary to add a lysing or detergent step (Pollard, D. R. et al., Rapid and specific detection of verotoxin genes in E. coli by the polymerase chain reaction, Journal of Clinical Microbiology, 28:540-545; Docherty, L. et al., The magnetic immunopolymerase chain reaction assay for the detection of Campylobacter in milk and poultry, Letters in Applied Microbiology, 22:288-292; Makino S et al., A new method for direct detection of Listeria monocytogenes from foods by PCR, Applied and Environmental Microbiology, 61:3745-3747). This lysing or detergent step will facilitate access to DNA in spores. Vegetative cells are easily lysed with a heating/microwaving procedure (G. H. Keller, M. M. Manak, DNA Probes, 2nd Edition, Stockton Press, New York, N.Y., pp. 1-659, 1993). No lysing or detergent step is necessary with many target pathogens, such as E. coli O157:H7.

### D. Detection of Amplified Sequences

In one embodiment, the detection of the amplification product is by gel electrophoresis, such as agarose gel electrophoresis. In another embodiment, the detection of the amplification product is by hybridization with a nucleic acid probe or probes. The hybridization can be solid phase or liquid phase. The hybridization can be dot hybridization with a nucleic acid probe or probes. The dot hybridization can be a radioactive dot blot or non-radioactive dot blot. The dot hybridization can be a dot blot micromethod or other method.

In another embodiment, the detection of the amplification product is by real time fluorescence measurement. In another embodiment, the detection of the amplification product is by immunoenzymatic assay using anti-RNA:DNA hybrid antibodies. In another embodiment, the detection of the amplification product is by nucleic acid probe assay using a fluorescein dye and/or fluorescent dye photometry. In another embodiment, the detection of the amplification product is by use of immobilized nucleic acid probes. Examples of substrates upon which the nucleic acid probes can be immobilized include, but are not limited to, a microplate, a membrane filter, a tube or a microchip. In one embodiment, a DNA probe is bound to a microwell plate. A labeled amplification product is then allowed to bind the DNA probe. The amplification product can be labeled, for example, with biotin, a colorimetric probe or a fluorescent dye. The presence of amplification product can then be detected by detection of the label, or where applicable, by detection of a secondary label that binds to or reacts with the labeled amplification product, such as horseradish peroxidase, alkaline phosphatase, labeled anti-biotin antibody, avidin, avidin-biotin complex or avidin-streptavidin complex.

In another embodiment, the detection of the amplification product is by detection of a radioactive label associated with the nucleic acid probe. Examples of methods for detecting radioactive labels associated with nucleic acid probes include, but are not limited to, autoradiography and scintillation counting (G. H. Keller, M. M. Manak, DNA Probes, 2nd Edition, Stockton Press, New York, N.Y., pp. 1-659, 1993). In another embodiment, the detection of the amplification product is by detection of a non-radioactive label associated with the nucleic acid probe. Examples of methods of detecting non-radioactive labels associated with nucleic acid probes include, but are not limited to, calorimetric, fluorometric or dye reporter assays. In another embodiment, the detection of the amplification product is by DNA hybridization/reverse hybridization. In another embodiment, the detection of the amplification product is by RNA synthesis. Nucleic acid probes for use in the detection of amplified nucleic acid sequences can be labeled during or following oligonucleotide synthesis.

In one embodiment, the invention provides a method for the specific detection of Escherichia coli O157:H7 in a food sample. A first step of this method comprises incubating the food sample in a tryptic soy broth (TSB) at about 37° C. for a sufficient time to allow for detection of subsequently amplified nucleic acid sequences. In one embodiment, this incubation is for about 2 to about 4 hours. As a second step, the food sample is contacted with magnetic beads coated with an antibody that specifically binds Escherichia coli O157:H7. The material bound to the magnetic beads is then selected from the food sample and nucleic acid sequences specific to Escherichia coli O157:H7 are amplified by PCR. The amplified nucleic acid sequences are then detected, the presence of amplified nucleic acid sequences indicating the presence of Escherichia coli O157:H7 in the food sample.

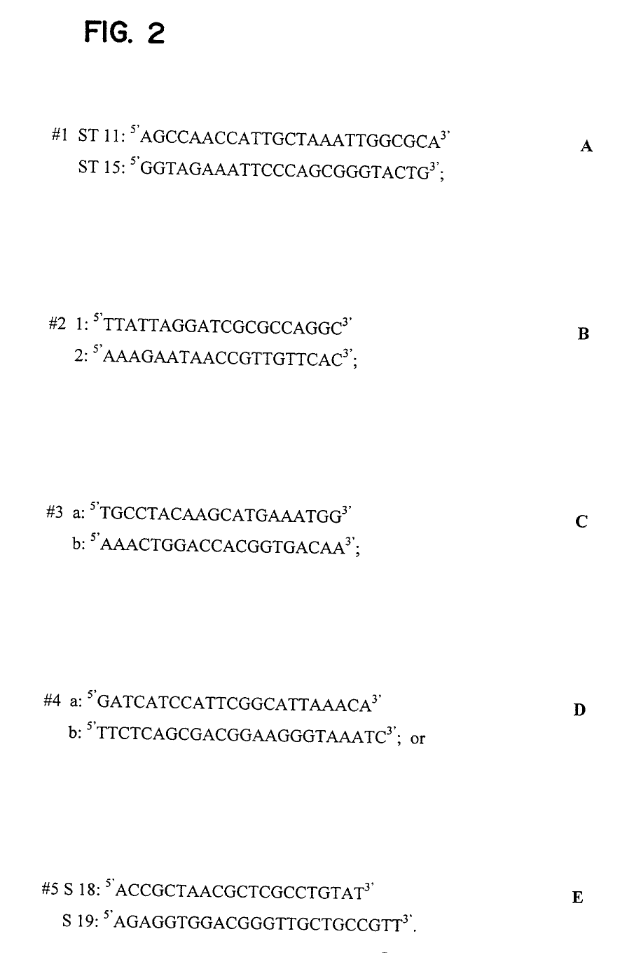

In another embodiment, the invention provides a method for the detection of Salmonella in a food sample. A first step of this method comprises incubating the food sample under sufficient conditions to culminate in a final titer suitable for subsequent detection of amplified nucleic acid sequences. As a second step, the food sample is contacted with magnetic beads coated with an antibody that specifically binds Salmonella. The material bound to the magnetic beads is then selected from the food sample and a set of primer sequences shown in FIG. 2 is used to amplify by PCR specific nucleic acid sequences recognized by these primers. The amplified nucleic acid sequences are then detected, the presence of the amplified nucleic acid sequences indicating the presence of Salmonella in the food sample. In one embodiment, the primer sequences amplified by PCR are those of set #1 shown in FIG. 2. In another embodiment, the primer sequences amplified by PCR are those of set #4 shown in FIG. 2.

Methods for each step of the invention: incubating samples, selecting for a target pathogen, amplifying nucleic acid sequences and detecting amplified nucleic acid sequences; are amenable to automation. Automation of any or all of the steps of the invention can further reduce the total assay time for detection of a target pathogen in a sample.

### Advantages of the Invention

The method of the invention permits the detection of a target pathogen with increased speed and increased specificity. Increased speed, or reduced detection time, is achieved through the unexpected synergistic effect of combining the incubation step with both immunoselection for the target pathogen and amplification of nucleic acid sequences specific to the target pathogen. Increased specificity is achieved through the use of a primer set that is specific for the target pathogen.

This invention is illustrated in the Experimental Details section which follows. This section is set forth to aid in an understanding of the invention but is not intended to, and should not be construed to, limit in any way the invention as set forth in the claims which follow.

### Experimental Details

### Example 1

### Detection of Escherichia coli O157:H7 in raw milk and ice cream

Escherichia coli O157:H7 (EHEC) in spiked samples of raw milk and ice cream was enriched in tryptic soy broth for 4 h, captured by immunomagnetic separation, subjected to amplification by polymerase chain reaction of parts of the verotoxin genes (SLT-I and SLT-II), and detected by agarose gel electrophoresis. Using this method, as few as one colony forming unit of E. coli O157:H7 per g of food could be detected in less than 10 h.

Our concept and strategy are based on the hypothesis that by a synergistic combination of the polymerase chain reaction (PCR) with both culture enrichment and immunomagnetic separation (IMS) it should be possible to detect EHEC rapidly and reliably, in the background of other microbes occurring in raw milk and milk products. Accordingly, we have optimised the culture enrichment, IMS and PCR procedures and incorporated all of them as integral steps in a new method that offers the necessary sensitivity and specificity to detect the presence of a single colony forming unit (cfu) of E. coli O157:H7 in 1 g of food, in less than 8 h.

Strains

Escherichia coli O157:H7 strains used in this study, their serotype, shiga-like toxin gene status, source and the result of the polymerase chain reaction using primers to the shiga-like toxin gene are presented in Table 1.

TABLE 1
Escherichia coli O157:H7 Strains

| UCD | SLT | | |
|---|---|---|---|
| Strain# | Genotype | Source | PCR Result |
| HF1 | I&II | ATCC 42895 | + |
| HF2 | I&II | ATCC 43894 | + |
| HF3 | None | ATCC 43888 | − |
| HF4 | II | ATCC 43889 | + |
| HF5 | I | ATCC 43890 | + |
| HF13 | I&II | CMDL | + |
| HF14 | I&II | CMDL | + |
| HF15 | I&II | CMDL | + |
| HF51 | II | USUHS | + |
| HF52 | I&II | USUHS | + |
| J5* | None | NK | − |

*Serotype 0111

UCD, University of California Davis (Calif., USA) Strain Number; SLT genotype, form of shiga-like toxin gene found in the strain; ATCC, American Type Culture Collection; CMDL, California Microbial Diseases Laboratory; USUHS, Uniformed Services University of Health Sciences; NK, Not Known. +, SLT-specific PCR product present; −, SLT-specific PCR product absent

Spiking of food samples with Escherichia coli O157:H7

E. coli O157:H7 was grown overnight in 50 ml of tryptic soy broth (TSB; Difco) in a 250 ml Erlenmeyer flask, shaking at 120 rpm at 37° C. Ice cream (Haagen-Dazs vanilla flavour, Teaneck, N.J. 07666, USA) was defrosted, aseptically unsealed and 45 g aliquots placed in 50 ml centrifuge tubes. The overnight culture was inoculated into and mixed with an ice cream sample, which was then diluted with the other ice cream samples in a ten-fold dilution series. Each dilution (200 ml) was plated onto MacConkey sorbitol agar (SMAC; March, S. B. & Ratman, S., 1986, Journal of Clinical Microbiology 23:869-872) containing cefimide (0.05 mg/l ) and potassium tellurite (2.5 mg/1) (SMACCT; Zadic et al., 1993) to estimate the numbers of E. coli O157:H7 present in the spiked ice cream samples. The ice cream samples were then mixed well and refrozen at −20° C., to simulate industrial storage conditions.

A decimal serial dilution of the overnight culture was made in TSB, and each dilution (1 ml) was mixed with 39 ml of raw bovine milk (UC Davis Dairy) in 50 ml centrifuge tubes. The spiked milk (200 ml) was then plated on SMACCT agar to obtain a plate count, and the remainder was stored overnight at 4° C., to simulate industrial storage and distribution practices.

Culture-Enrichment of the Pathogen

Raw milk (10 ml) or ice cream (10 g) was aseptically removed from each spiked sample and resuspended in 90 ml of TSB in 250 ml Erlenmeyer flasks. The cultures were then incubated with shaking at 120 rpm at 37° C. for 4 h.

Immunomagnetic Separation

After the 4 h incubation, 1 ml aliquots from each enriched culture were mixed with 20 μl of O157 antibody-coated magnetic beads (Dynabeads anti-E. coli O157:H7, Dynal As, Oslo, Norway) in a 1.8 ml microfuge tube. The tube was then shaken gently for 45 min, and the beads were washed according to the manufacturer's instructions and pelleted in a microfuge, in preparation for the PCR analysis.

Polymerase Chain Reaction

PCR primers were synthesised by the UC Davis Protein Structure Laboratory, using an Applied Biosystems automatic DNA synthesiser Model 380A. This primer set amplifies a 215 base pair (bp) coding sequence of the A subunit of SLT-I and a 212 bp coding sequence of the A subunit of SLT-II. Paton et al. (1993, Journal of Clinical Microbiology 31:3063-3067) have previously confirmed the E. coli O157:H7-specificity of this primer set in the PCR examination of human faecal cultures. The primer sequences are:

A: ^{5′}-ATACAGAG(GA)G(GA)ATTTCGT-^{3′}

B: ^{5′}-TGATGATG (AG)CAATTCAGTTAT-^{3′}.

The PCR amplification mix contained a final concentration of 10 mM Tris-HCl pH 9.0, 50 mM KCl, 1 ml/l Triton X-100, 1.8 mM MgCl₂, 200 μM each of the deoxynucleoside triphosphates (Pharmacia, Piscataway, N.J. 08855, USA), 1.5 units of Taq polymerase (Promega, Madison, Wis. 53711, USA) and 0.5 μM each of the primers, in a total reaction volume of 100 μl. The PCR mix was added to the beads and overlaid with 70 μl of mineral oil. The PCR amplifications were performed in a thermocycler (model PTC150, M. J. Research, Watertown, Mass. 02172, USA) with a 2 min initial denaturation at 94° C., followed by 35 cycles of 92° C. for 1 min, 48° C. for 1 min and 72° C. for 1 min. At the end of the PCR, 10 μl from each reaction was analysed by electrophoresis for 30 min at 100 V in a 1.5% agarose gel (containing 0.5 μg/ml ethidium bromide) in Tris-acetate buffer (TAE; Sambrook, J., Fitch, E. F & Maniatis, T., 1989, Molecular cloning a laboratory manual. 2nd edn., Cold Spring Harbor, N.Y. Cold Spring Harbor Laboratory Press) containing 0.5 μg/ml ethidium bromide. HaeIII-digested fX174 DNA was used as a size marker. The DNA bands in the gels were visualised by UV transillumination and photographed.

The detection of Escherichia coli O157:H7 in spiked ice cream

The fidelity of the primers to both forms of the SLT gene was established by testing 5 μl aliquots of the enriched cultures of all E. coli strains (Table 1) in the PCR. PCR products of the expected size (212-215 bp) for the SLT genes were obtained with all the strains tested, with the exception of J5 and HF3 both of which do not carry either of the SLT genes.

Before spiking with the test pathogen we wanted to ensure that the ice cream was free from prior contamination with it. Samples (10 g) inoculated into 90 ml TSB, incubated at 37° C. for 16 h and 0.2 ml aliquots spread onto SMAC showed no contamination with E. coli. The ice cream was then spiked with E. coli O157:H7 at various contamination levels and frozen at −20° C. for at least 24 h, to freeze-stress the bacteria. The ice cream was then defrosted, enriched and used for IMS-PCR as described above. Plate counts of samples of the ice cream obtained on SMACCT before and after freezing showed no measurable change as a consequence of freezing.

Contamination levels of strains HF1, HF4 and HF5 ranging between zero and 10⁸ cfu/g of ice cream were used in validation of the procedure. In all cases when the initial inoculum was 1 cfu/g or greater a PCR product of the expected size for SLT genes was obtained, demonstrating the high sensitivity of the method in the detection of E. coli O157:H7. The unspiked samples did not give a PCR product. Positive control PCR amplifications containing E. coli O157:H7 strain HF1 cells and negative control PCR amplifications containing no cells, consistently tested positive and negative, respectively.

The detection of Escherichia coli O157:H7 in spiked raw milk

No evidence of prior contamination with E. coli was found when samples (0.2 ml) of raw milk were plated directly on SMAC agar. However, when samples were plated on SMAC agar after enrichment for 4 h in TSB, low levels (˜1 cfu/ml) of sorbitol-positive E. coli were detected, indicating low-level faecal contamination of raw milk samples with non-O157:H7 E. coli.

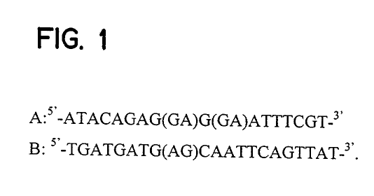

The milk samples were then inoculated with serial dilutions of E. coli O157:H7 and stored at 4° C. for at least 16 h, to cold-stress the bacteria. Plate counts of E. coli O157:H7 in the milk samples on SMAC agar before and after the cold-treatment showed no measurable reduction indicating that the storage conditions had no effect on E. coli O157:H7 viability. The results of a validation experiment using strain HF1 are presented in FIG. 1. With contamination levels ranging from 4 to 3×10⁵ cfu/ml of milk, all the spiked samples tested PCR-positive, but none of the unspiked. Further experiments using strains HF1, HF52, HF4 and HF5 at 1 cfu/ml or greater consistently gave SLT-positive PCR products.

Samples of the TSB enrichment culture were also plated out before and after the 4 h enrichment at 37° C. to assess the efficiency of the enrichment procedure and to get an estimate of the actual numbers of bacteria being detected by the IMS-PCR procedure. The results as presented in Table 2, showed that the 4 h enrichment caused a 200-900 fold increase in the cell numbers of E. coli O157:H7 and that 800 cfu were sufficient to be detected by the IMS-PCR procedure.

TABLE 2
Escherichia coli O157:H7 Strain HF2 Before and After Enrichment;
Detection by PCR

| No. of E. coli/ml | No. of E. coli/ml | |
|---|---|---|
| before enrichment (cfu) | after enrichment (cfu) | PCR product |
| 370 | 9 × 10⁴ | + |
| 34 | 1 × 10⁴ | + |
| 0.9 | 8 × 10² | + |
| 0 | 0 | − |

PCR, polymerase chain reaction; cfu, colony forming units.
+, SLT-specific PCR product present; −, SLT-specific PCR product absent

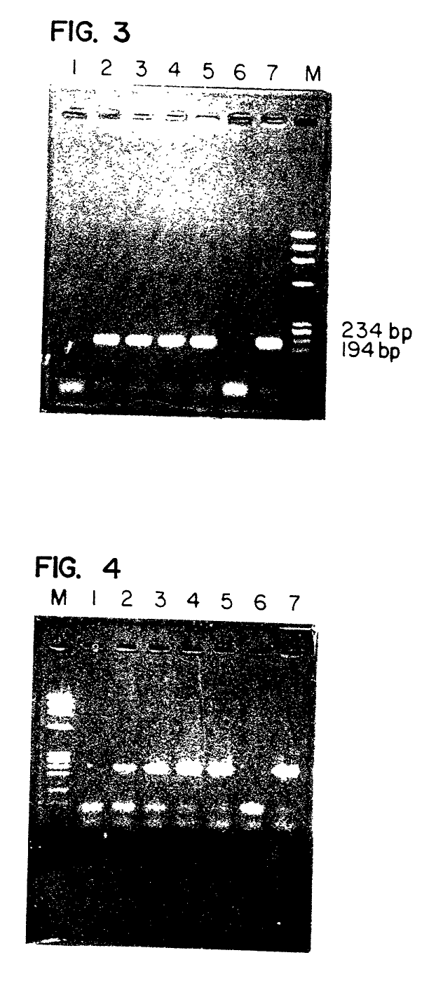

FIG. 3 shows the results of gel electrophoresis of samples following the 4 hour enrichment followed by immunomagnetic separation to remove E. coli O157:H7 cells and PCR amplification of fragments of the shiga-like toxin genes. Lane 1 shows unspiked sample; lane 2, sample spiked with 4 cfu/ml; lane 3, sample spiked with 36 cfu/ml; lane 4, sample spiked with 4×103 cfu/ml; lane 5, sample spiked with 3×105 cfu/ml; lane 6, negative control, no DNA or cells added; lane 7, positive control containing strain HF1 cells. Lane M is φX174 DNA marker digested with HaeIII (the 234 bp and 194 bp markers are noted to the right).

Rapid and reliable detection of pathogens is of critical importance to the development of effective control and prevention of foodbome diseases. In an effort to complement the existing pathogen detection methods used in the dairy industry with modern techniques offering increased speed, specificity and sensitivity, we have developed a novel method by combining appropriate sections of different microbiological and molecular biological procedures used in molecular diagnostics. Using the new method we have demonstrated the feasibility of detecting E coli O157:H7, contaminating milk and ice-cream, at levels as low as one cfu per unit (g or ml) of each food sample, within 8 h.

Enriching in non-selective media increases the likelihood of detecting bacteria that have been stressed chemically, mechanically, or by temperature during manufacturing processes. Normally a selective agent such as novobiocin, cefimide or potassium tellurite is included in the enrichment broth to reduce the numbers of competing bacteria and to allow enrichment of the target bacteria. However, we found that the inclusion of novobiocin in the enrichment broth prevented the growth of E. coli O157:H7 when the cells had been cold-stressed (4° C. for 4 days in TSB). Our observation corroborates the finding of Mackey (Mackay, 1988, FEMS Microbiology Letters 20:395-399) that various selective agents, including novobiocin, reduce the viability of E. coli cells subjected to physical stress. As we only required a short enrichment step, and washing and collection of target bacteria was achieved using IMS, the benefits of using a non-selective enrichment media outweighed those of using a selective media.

In sharp contrast to the currently used detection methods requiring initial enrichment of up to 24 h (Bennett, A. R., Macphee, S. & Betts, R. P., 1995, Letters in Applied Microbiology 6:375-920), we found that a 4 h enrichment, giving an estimated 9 doublings of E. coli O157:H7, produces sufficient cells from 1 cfu to allow detection by IMS-PCR.

In food-related applications, IMS is predominantly used for the separation of microbial cells from the food matrix and non-target microbes (Okrend, A. J. G., Rose, B. E. & Lattuada, C. P., 1992, Journal of Food Protection 55:214-217; Wright, D. J., Chapman, P. A. & Siddons, C. A., 1994, Epidemiology and Infection 113:31-39). Microscopic paramagnetic particles coated with pathogen-specific antibodies concentrate the target cells from the enrichment broth. We used magnetic beads in our procedure as a selective enrichment step to capture E. coli O157:H7. The IMS step, in addition to selectively concentrating the target microbe from a heterogeneous microbial population present in the test food samples, also reduces potential interference from food ingredients by separating the bacteria from the food homogenate. This is important because although PCR is a robust method, there have been reports of food components interfering with the PCR (Powell, H. A., et al., 1994, Letters in Applied Microbiology 18:59-61). Our results, using IMS, showed no evidence for PCR inhibition by the food matrix. However, we observed that the capture of the E. coli onto the immunomagnetic beads was not 100% efficient because up to 25% of them were lost during the washing step.

The synthesis of a PCR product of the correct size serves as a quick and definitive confirmation of the identity of the target organism. The fidelity and target-specificity of the primers determine the confidence level of the PCR procedure for the confirmation of the pathogen identity. A number of primer sets with specificity to EHEC, e.g., those based on the SLT-I and SLT-II genes, have been designed and successfully used by others to detect EHEC from a variety of matrices, e.g., faeces, ground beef (Paton, et al., 1993, Journal of Clinical Microbiology 31:3063-3067; Gannon, V. P. J., et al., 1992, Applied and Environmental Microbiology 58:3809-3815; Samadpour, M., et al., 1990, Applied and Environmental Microbiology 56:1212-1215). Using one of these primer sets we have demonstrated detection of low levels of E. coli O157:H7 in milk and ice cream.

With the increasing requirement for food producers to demonstrate “Due Diligence”, there is a need for rapid detection methods. Our method can detect one bacterial cfu present in the test food sample, in less than one working day. The speed and sensitivity offered by our method could benefit dairy producers as well as food inspectors, since the methodology is not technically demanding and can be easily and rapidly performed by technical personnel with minimal training. Further, the method has been designed to keep the implementation steps to a minimum, not requiring selective enrichment nor DNA isolation, facilitating future automation. Our method, in its current format, using agarose gel electrophoresis as the end-point detection is very sensitive, rapid and simple for the detection of E. coli O157:H7 in dairy products, and has been directly applicable to meat samples and to other foodborne bacterial pathogens (see Examples 2 and 3). Nevertheless, emerging signal-amplification detection technologies, such as fluorescent dyes and laser detection, may increase the sensitivity of the method and consequently reduce the detection time even further.

### EXAMPLE 2

### Detection of Escherichia coli O157:H7 in Ground Meat

An immuno-polymerase chain reaction in conjunction with pre-enrichment was performed for rapid and reliable detection of Escherichia coli O157:H7 in ground beef. The E. coli 0 1 57:H7 cells were separated quickly from the enrichment culture, using magnetic beads coated with E. coli O157:H7-specific antibody; the bead-bound bacterial cells were subjected to polymerase chain reaction (PCR) to amplify specific segments of the SLT-I and SLT-II genes of E. coli O157:H7, and the amplified DNA was detected by agarose gel electrophoresis. Detection of a single colony forming unit (cfu) of E. coli O157:H7 was achieved in 8 h.

Strains

Table 3 lists the bacterial strains used in the study. Counts of E. coli O157:H7 were obtained by plating on SMACCT (Zadik, P. M., Chapman, P. A. & Siddons, C. A., 1993, Journal of Medical Microbiology. 39:155-158)) agar plates.

TABLE 3
Bacterial strains used, SLT gene status and sources

| UCD | | SLT | Orignial |
|---|---|---|---|
| Number | Strain | Genotype | Source |
| HF1 | Escherichia coli O157:H7 | 1&2 | ATCC 42895 |
| HF2 | E. coli O157:H7 | 1&2 | ATCC 43894 |
| HF3 | E. coli O157:H7 | 0 | ATCC 43888 |
| HF4 | E. coli O157:H7 | 2 | ATCC 43889 |
| HF5 | E. coli O157:H7 | 1 | ATCC 43890 |
| HF13 | E. coli O157:H7 | 1&2 | CMDL |
| HF14 | E. coli O157:H7 | 1&2 | CMDL |
| HF15 | E. coli O157:H7 | 1&2 | CMDL |
| HF51 | E. coli O157:H7 | 2 | USUHS |
| HF52 | E. coli O157:H7 | 1&2 | USUHS |
| J5 | E. coli O111 | 0 | NK |
| CG1 | Aeromonas hydrophila | 0 | ATCC 7965 |
| CG2 | Bacillus cereus | 0 | ATCC 14579 |
| CG3 | Citrobacter freundii | 0 | ATCC 8090 |
| CG4 | Enterobacter cloacae | 0 | ATCC 13047 |
| CG5 | Enterococcus faecalis | 0 | ATCC 19433 |
| CG6 | Hafnia alvei | 0 | ATCC 29926 |
| CG7 | Klebsiella pneumoniae | 0 | ATCC 13883 |
| CG8 | Proteus vulgaris | 0 | ATCC 13315 |
| CG9 | Pseudomonas aeuoginosa | 0 | ATCC 10145 |
| S36 | Salmonella enteritidis | 0 | ATCC 13076 |
| CG10 | Shigella sonnei | 0 | ATCC 29930 |
| CG11 | Staphylococcus aureus | 0 | ATCC 12600 |

ATCC, American Type Culture Collection; CMDL, California Microbial Diseases Laboratory; USUHS, Uniformed Services University of Health Sciences; NK, Not Known; 0, SLT gene(s) absent.

Experimental contamination of ground beef samples with E. coli O157:H7

Overnight cultures of E. coli O157:H7 were grown in tryptic soy broth (TSB; Difco) in Erlenmeyer flasks, shaking at 120 rpm at 37° C. Ground beef was divided under aseptic conditions into 25 g portions, individually rolled into balls and laid out in an alcohol-sterilized plastic box. Each meat ball was then injected with an aliquot (100 μl) each of serial dilutions (10 to 10⁹) of an overnight culture in TSB, using a pipetman (P-200) tip. The inoculated meatballs were left at room temperature for 1 h, to allow the injected liquid to be absorbed, and then frozen at −20° C. for at least 24 h.

Enrichment of the bacteria present in the ground meat

The frozen meatballs were each transferred to the mesh insert of a stomacher bag (Applied Biosolutions, New York), and were thawed at room temperature for 30 min. Then, 225 ml of TSB were added to each bag, and the meatballs were homogenized for 1 min in a Stomacher (Colworth). The meat homogenate was then incubated in the bag at 37° C. for 4 h.

Immunomagnetic Separation (IMS) of E. coli O157:H7

After the 4-h incubation step, aliquots (1 ml) of each enriched culture were removed from the outer compartment of the stomacher bag and mixed with 20 μl of O157:H7 antibody-coated magnetic beads (Dynabeads-anti E. coli O157:H7, Dynal AS, Oslo, Norway), in a 1.8-ml microfuge tube. The tube was rocked gently for 30 min, and the beads were recovered using a magnetic particle concentrator (Promega), washed according to the manufacturer's instructions and pelleted by centrifugation for 1 min at 14,000×g, in preparation for the PCR analysis.

Amplification of the SLT-I and/or SLT-II gene(s) by PCR

The PCR amplifications were carried out as described in Example 1, using a primer set derived from the Shigella-like toxins, SLT-I and SLT-II. The primers, 5′-ATACAGAG(GA)G(GA)ATTTCGT-3′ and 5′-TGATGATG(AG)CAATTCAGTAT-3′, amplify the 215-bp and the 212-bp segments encompassing the 586-800 bp of the SLT-I coding sequence and the 583-794 bp of the SLT-II coding sequences, respectively. Paton et al., (1993, Journal of Clinical Microbiology 31:3063-3067) designed these primer sequences and demonstrated their E. coli O157:H7-specificity in the PCR examination of faecal cultures of infected humans.

The PCR buffers (100 μl reaction volumes) containing final concentrations of: 10 mM Tris-HCl, pH 9.0, 50 mM KCl, 1.8 mM MgCl₂, 1 ml/ L Triton X-100, 200 μM each of the deoxynucleoside triphosphates (Pharmacia), 1.5 U of Taq polymerase (Promega) and 0.5 μM each of the primers, were added to the immunomagnetic beads (20 ul), mixed and then transferred to a PCR tube and overlaid with 70 μl of mineral oil. The amplifications were performed in a thermocycler (M. J. Research, model PTC150) with a 2-minute initial denaturation at 94° C., followed by 35 cycles of denaturation at 92° C. for 1 min, primer annealing at 48° C. for 1 min and extension at 72° C. for 1 min. The products (10 μl) from each reaction were resolved by electrophoresis for 30 min at a constant power of 100 V, in a 1.5% agarose gel in Tris-actate-EDTA (TAE) buffer, both containing 0.5 μg/ml ethidium bromide. HaeIII digest of fX 174 DNA was used as DNA size markers. The DNA bands in the gels were illuminated by UV light and were photographed.

The principal goal of our method is to reduce the overall detection time, without compromising the specificity or sensitivity. We have accomplished this goal by a combination of the steps: pre-enrichment of the pathogen, selective capture of the pathogen using IMS, amplification of the pathogen-specific DNA sequences using PCR, and detection of the amplified DNA by agarose gel electrophoresis. The total assay time can be considerably reduced when these steps are used together rather than individually. Accordingly, we first optimized each of these steps and then integrated them into a single method.

Pre-enrichment of the culture

The pre-enrichment step helps by increasing the bacterial cell number to the minimum detection threshold. In the past, this step has been carried out by others for considerably long periods, ranging from 6 to 48 h, using selective media that promote the growth of the target pathogen and suppress the growth of non-target organisms. However, such media can impede the recovery of stressed organisms, including the target organism. The use of a non-selective enrichment allowed us to enrich E. coli O157:H7 (with a doubling time of 26 min) within 4 h from a single cfu to 500 cfu, the minimal number of target bacterial cells needed for ensuring the specificity and sensitivity of the subsequent PCR step. It did not appear that the growth of E. coli O157:H7 was affected by competing organisms, possibly due to the short enrichment time used at this stage.

Efficiency of immunomagnetic separation

IMS, by offering a considerably faster means of collecting cells than centrifugation, saves a lot of time in isolating, washing, collecting and concentrating bacterial cells from food samples. More importantly, it eliminates matrix effects, including potential PCR-inhibitors from food and enrichment media. For these advantages, we have chosen to optimize and use IMS in our method.

We have found a 30 min-incubation of the beads with enrichment broth (1 ml) containing about 28 cfu/ml to yield a PCR product of the same intensity as that obtained after 45 or 60 min, while no product is obtained from the sample incubated for 15 min. Therefore, we used 30-min as the standard incubation period.

We then determined the capacity and saturation limits of the IMS beads in recovering the target organism, E. coli O157:H7 from the broth, containing between 10² and 10⁹ cells. We found that the IMS beads (20 ml) could recover 80% of the total bacterial population from the enrichment culture containing 10⁹ cfu, 95% of 10⁷ cfu, over 99% of 10⁴, and 100% of 10² cfu/ml. It is clear from these results that the bacterial recovery efficiency of the immunomagnetic beads is limited to the binding sites available on their surface. Although 20 ml of the IMS beads have the surface area sufficient to capture between 10⁷ and 10⁸ cfu, 500 cfu of E. coli O157:H7 are all that is needed for PCR. This offers the possibility to either limit the length of the enrichment step to culminate in a final titer of 500 cfu, or for reduction in the quantity of the beads used.

Optimization of PCR conditions

To determine the optimal conditions for PCR, we first investigated the influence of magnesium concentration on PCR, by carrying out a magnesium titration experiment. When magnesium chloride (MgCl₂) concentrations were varied between 0.5 and 2.5 mM, with 1 to 100 target bacterial cells as the template source, an amplified DNA band of the expected size and intensity was obtained with all concentrations of MgCl₂ above 1 mM, but the band-intensity was highest in the 1.5 to 2.5 mM range. Hence, we used 1.5 mM MgCl₂ in all subsequent assays.

We also investigated the effect of the number of cycles on the sensitivity of PCR, by examining reaction mixes containing between 2 and 165 cells in each of 30, 40 and 50 cycles. The results showed a lower limit of 18 cfu with a 30-cycle reaction, and 2 cfu with a 40 cycle reaction. After 50 cycles, there was evidence of a second smaller band. It thus appears desirable to keep the number of cycles to a minimum, since too many cycles can allow the products of non-specific primer binding to become noticeable, as above. With further evidence from a different set of experiments that enabled detection of 1 cfu of E. coli O157:H7 using a 35-cycles PCR, the number of cycles was fixed at 35 for all subsequent experiments.

The specificity of the primers was ascertained by testing them against a panel of target and non-target bacterial strains (Table 3). PCR products of the expected size were only obtained with E. coli strains of the O157:H7 serotype, carrying either copy of the SLT genes (I or II), but not with E. coli of any other serotype nor with different species of the non-E. coli bacteria tested (FIG. 1).

The results of electrophoretic detection of the SLT-gene amplification products from the immuno-PCR of ground beef samples, spiked with Escherichia coli O157:H7 are presented in FIG. 4. Ground beef samples spiked with E. coli O157:H7, strain HF12 were used for lanes 2 to 6 following 4 h enrichment, removal of the E. coli O157:H7 cells by immunomagnetic separation, and PCR amplification of fragments of the Shiga-like toxin genes. The appearance of a 212/215 base pair band on a 1.5% agarose gel was taken as the indication of contamination. Lane M, DNA size markers: fX 174 Hae III digest; Lanes 2-8, samples: lane 2, unspiked; lane 3 spiked with 2 colony forming units (cfu)/g; lane 4, spiked with 30 cfu/g; lane 5, spiked with 400 cfu/ml; lane 6 spiked with 3×10⁴ cfu/g; lane 7, negative control, no DNA or cells added; lane 8; positive control, containing HF1 cells.

Sensitivity of the method

Table 4 shows the effect of the decreasing levels of HF2, HF4 and HF14 on the sensitivity of the new method. Detection of 1 cfu of E. coli O157:H7 per g of meat was achieved in all the cases, using the protocol we have devised, regardless of the strain variations. One sample of beef, however, tested positive by PCR, even without spiking, indicating either a possible prior contamination of the meat at one of the source points or that the primers were detecting cross-reacting species of bacteria. Examination of additional samples from the same batch of beef by PCR and spread plating on SMACCT agar revealed prior contamination of this batch of beef with ETEC, leading to the rejection of this meat as well as the results obtained by its use. Nonetheless, the observation demonstrated the ability of the method for detecting ETEC in “naturally” contaminated meat samples.

TABLE 4
Sensitivity limits of the immuno-PCR procedure for detecting 3
different strains of Escherichia coli O157:H7 in ground beef

| CFU/g | Strain | | |
|---|---|---|---|
| Ground beef | HF14 | HF4 | HF5 |
| >10,000 | + | + | + |
| 1,000-10,000 | + | + | + |
| 100-1,000 | + | + | nt |
| 10-100 | + | + | + |
| 1-10 | + | + | + |
| <0.1, >1 | nt | + | − |
| 0 | − | − | − |

Cfu, colony forming units; +, PCR reaction positive; −, PCR reaction negative; nt, not tested.

In conclusion, by optimizing and appropriately combining the steps of pre-enrichment, IMS and PCR, followed by detection of the signature-sequences of the pathogen, we have created a rapid method with the sensitivity to selectively detect as few as 1 cfu of E. coli O157:H7 in 1 g of ground meat, within 8 h (or less, in meat with higher levels of contamination). The method has broad application to a wide range of microbial pathogens, after minimal modifications. The experimental conditions that may need target-specific but standard alterations for its application for detection of a new target pathogen include: the template sequence, the primers, optimal PCR conditions for target sequence amplification, antibody reagent(s) when the IMS step is included, and media and culture conditions if the enrichment step is used.

### EXAMPLE 3

### Primer-fidelity for PCR-detection of Salmonella

In an effort to extend the application of our method to Salmonella detection, we first surveyed the literature for published oligonucleotide primer sets used for PCR-detection of Salmonella, and chose 5 of them for our investigation (Aabo, S. et al., 1993, Molecular and Cellular Probes 7:171-178; Fluit, A. C. et al., 1993, Appl. Env. Microbiol. 59:1342-1346; Stone, G. G. et al., 1994, J. Clin. Microbiol. 32:1742-1749; Jitrapakdee, S. et al., 1995, Molecular and Cellular Probes 9:375-382; Kwang, J.; Littledike, E, T. & Keen, J. E., 1996, Lett. Appl. Microbiol. 22:46-51). We have now synthesized all 5 of these primer sets and assessed their fidelity and utility in PCR, with 15 Salmonella strains: S. agona; S. anatum; S. enteritidis; S. havana; S. krefeld; S. lilee; S. melegredis; S. montevideo; S. munster; S. newport; S. saintpaul; S. schwarzengrund; S. tennessee; S. typhimurium and S. worthington, and 13 non-Salmonella strains: Aeromonas hydrophila; Bacillus cereus; Citrobacter freundii; Enterobacter cloacae; Entero. faecalis; E. coli non-VTEC; Hafnia alvei; Klebsiella pneumoniae; Proteus vulgaris; Pseudomonas aeroginosa; Shigella sonnei; Staphylococcus aureus and Yersinia enterocolitica. The PCR product was analyzed as a function of the band position (as an indication of the amplified DNA fragment size) on an agarose gel, and its intensity as well as its production as single or multiple DNA bands. The results are presented here and used as the basis for evaluation of the merits of each primer set.

Overnight culture of E. faecalis was grown in Brain Heart Infusion broth (BHIB; Difco) and all the other bacteria in Tryptose Soy Broth (TSB; Difco), at the appropriate temperatures. The PCR mixes were made up using identical concentrations of primers, dNTPs and MgCl₂ as specified in the corresponding papers, and the Taq polymerase and the buffer obtained from Promega. The primer sets consisted of:

#1 ST 11: ^{5′}AGCCAACCATTGCTAAATTGGCGCA^{3′}/

ST 15: ^{5′}GGTAGAAATTCCCAGCGGGTACTG^{3′};

#2 1: ^{5′}TTATTAGGATCGCGCCAGGC^{3′}/

2: ^{5′}AAAGAATAACCGTTGTTCAC^{3′};

#3 a: ^{5′}TGCCTACAAGCATGAAATGG^{3′}/

b: ^{5′}AAACTGGACCACGGTGACAA^{3′};

#4 a: ^{5′}GATCATCCATTCGGCATTAAACA^{3′}/

b: ^{5′}TTCTCAGCGACGGAAGGGTAAATC^{3′};

#5 S 18: ^{5′}ACCGCTAACGCTCGCCTGTAT^{3′}/

S 19: ^{5′}AGAGGTGGACGGGTTGCTGCCGTT^{3′}.

An aliquot (5 ml) of the overnight culture of each strain was subjected to PCR amplification. The PCR amplifications were performed in a thermocycler (M. J. Research, model PTC150) using the conditions outlined in the original papers. Negative controls using TSB (5 ml) were included with each set of amplifications. The accuracy of the temperature profiling of the thermocycler had previously been confirmed using an independent thermocouple linked to a digital thermometer. The reactions (10 μl of each reaction) were analyzed by electrophoresis for 30 min at 100 V in agarose (1.5%) gel in Tris-acetate-EDTA (TAE) buffer, both containing 0.5 μg/ml ethidium bromide. HaeIII-digested fX174 DNA was used as size marker. The DNA bands in the gels were visualized by UV transillumination and photographed.

We found that although all 5 primer sets can detect all the Salmonella strains tested, none of them is specific to a broad range of test strains, probably because each primer set was standardized against an entirely different set of Salmonella strains. A problem shared by all primers is the production of non-specific PCR product with Shigella. The PCR product with primer set #4 with Shigella gives a band on agarose gel at exactly the same position as with Salmonella. Further, all of the primers, except set #4 produced multiple non-specific bands of non-target size, in addition to the target product, in a few instances (Table 5).

TABLE 5
Summary of PCR primers, their properties and results of use in PCR-
detection of Salmonella

| | | | Expected Salmonella | | | | |
|---|---|---|---|---|---|---|---|
| | | | size of strains | | | | |
| | | | PCR | | | Non-Salmonella | |
| Primer | Template | | product, | | | spp. Tested | |
| set, # | sequence | Reference | bp | Total | PCR + ve | Total | PCR + ve |
| 1 | “Salmonella- | Molecular | 429 | 146 | 144 | 86 | 0 |
| | specific | & | | | | | |
| | sequence” | Cellular | | | | | |
| | | Probes | | | | | |
| | | 7:171 | | | | | |
| | | (1993) | | | | | |
| 2 | oriC | Appl. Env. | 163 | 2 | 2 | 0 | |
| | | Microbiol. | | | | | |
| | | 59:1342 | | | | | |
| | | (1993) | | | | | |
| 3 | invA, InvE^{a} | J. Clin. | 457 | 47 | 47 | 53 | 2 |
| | | Microbiol. | | | | | |
| | | 32:1742 | | | | | |
| | | (1994) | | | | | |
| 4 | Repeat | Molecular | 199 | 52 | 52 | 9 | 0 |
| | sequence | & | | | | | |
| | | Cellular | | | | | |
| | | Probes 9: | | | | | |
| | | 375 | | | | | |
| | | (1995) | | | | | |
| 5 | ompC^{b} | Lett. Appl. | 159 | 40 | 40 | 24 | 0 |
| | | Microbiol. | | | | | |
| | | 22:46 | | | | | |
| | | (1996) | | | | | |

^{a}Required confirmation with internal primers/probes in the case of Y. pseudotuberculosis and Edwardsiella tarda.
^{b}Required confirmation by Southern blotting and probe hybridization.

Primer set #2 failed to yield PCR product with 2 different strains of S. enteritidis, although all the rest of the primer sets elicited PCR products of the expected size with these strains. Primer set #5 produced multiple non-specific bands of non-target size, in addition to the target product even with Salmonella strains, i.e., S. lilee and S. melegredis. This inconsistency decreases the level of confidence of detection. Thus, of all the primers we tested, set #4 consistently showed reliable sensitivity, reproducibility and specificity. Primer set #1 also appears to be amenable to optimization and use with reliable results.

The above discrepancies, at least to an extent, may be attributed to limitations inherent to the PCR technique, including variations caused by the polymerase and the buffer, the make and model of the PCR equipment. Another likely source of bias may be our direct use of whole bacteria as the template source in variance to the use of isolated DNA by the previous authors. In light of these results together with the limited characterization of the end-product of the assay, viz., agarose gel electrophoretic visualization of the gross PCR product, a quick ‘yes’ or ‘no’ answer could be obtained by PCR cautious use of primer set #4 or #1, contingent upon a simultaneous verification for cross-reactivity to Shigella (e.g., a Shigella-specific PCR), until alternative solutions can be found by way of better-designed primers for PCR (which could come from genome projects) or of improved primer-independent procedures that could match the speed and sensitivity of PCR.

TABLE 6
Gel electrophoretic profile obtained using primers designed for PCR-
detection of Salmonella

| | PCR-product using primer set | | | | |
|---|---|---|---|---|---|
| Bacterial Strain | #1 | #2 | #3 | #4 | #5 |
| Aeromonas hydrophila | o | n | o | o | n |
| Bacillus cereus | o | o | o | o | o |
| Citrobacter freundii | m,f | n | n | o | n |
| Enterobacter cloacae | o | n | n | o | m |
| Enterococcus faecalis | o | o | o | o | o |
| Escherichia coli non-VTEC | o | o | o | o | o |
| Hafnia alvei | n,f | n | n | o | n |
| Klebsiella pneumoniae | o | n | n | o | o |
| Proteus vulgaris | n | n | o | o | o |
| Pseudomonas aeroginosa | o | n | o | o | m |
| Shigella sonnei | n,f | n | n | s | n |
| Staphylococcus aureus | o | o | o | o | o |
| Yersinia enterocolitica | n,f | n | n | o | n |

PCR products obtained with the 5 different primer sets with 13 non-Salmonella bacterial strains: f, faint band; m, multiple PCR bands including Salmonella-positive sized band; n, PCR band/bands of non-target size; o, no PCR band; s, single Salmonella-positive PCR band; −, not tested.

We claim:

1. A method for the specific detection of a target pathogen in a sample, the method comprising:
(a) incubating the sample under sufficient conditions to enrich the target pathogen population in the sample;
(b) immunoselecting the target pathogen from the sample enriched in step (a);
(c) amplifying nucleic acid sequences specific to the target pathogen from the target pathogen so selected in step (b); and
(d) detecting the nucleic acid sequence so amplified in step (c), the presence of the amplified nucleic acid sequence indicating the presence of the target pathogen.

2. The method of claim 1, wherein the target pathogen is a gram-negative enterobacterium.

3. The method of claim 2, wherein the target pathogen is E. coli.

4. The method of claim 3, wherein the target pathogen is E. coli O157:H7.

5. The method of claim 1, wherein the target pathogen is Salmonella.

6. The method of claim 6, wherein the Salmonella is selected from S. agona; S. anatum; S. enteritidis; S. havana; S. krefeld; S. lilee; S. melegredis; S. montevideo; S. munster; S. newport; S. saintpaul; S. schwarzengrund; S. tennessee; S. typhimurium and S. worthington.

7. The method of claim 1, 2, 3, 4, 5 or 6, wherein in step (b) immunoselecting the target pathogen is by immunomagnetic separation.

8. The method of claim 7, wherein immunomagnetic separation comprises contacting the sample with magnetic beads coated with an antibody or fragment thereof that specifically recognizes, binds and captures the target pathogen.

9. The method of claim 1, 2, 3, 4, 5, 6, 7 or 8, wherein the amplification of step (c) is by PCR.

10. The method of claim 1, 2, 3, 4, 5, 6, 7, 8 or 9, wherein the detecting of step (d) is by electrophoresis.

11. A method for the specific detection of Escherichia coli O157:H7 in a food sample, the method comprising:
(a) incubating the food sample in culture medium under conditions so that Escherichia coli O157:H7 is enriched;
(b) contacting the food sample of step (a) with an antibody that recognizes and binds Escherichia coli O157:H7 so as to form an antibody/Escherichia coli O157:H7 complex;
(c) selecting the complex from the food sample of step (b);
(d) amplifying the nucleic acid sequences shown in FIG. 1 from the complex selected in step (c); and
(e) detecting the nucleic acid sequences so amplified in step (d), the presence of the amplified nucleic acid sequences indicating the presence of the target pathogen.
